(19) United States
(12) Patent Application Publication
(10) Pub. No.: US 2025/0241545 A1
(43) Pub. Date: Jul. 31, 2025

(54) SPHYGMOMANOMETER

(71) Applicant: OMRON HEALTHCARE CO., LTD., Muko-shi (JP)

(72) Inventors: Jun NAKATSUKA, Kyoto (JP); Daisuke ISHIHARA, Kyoto (JP); Kohei HAYAKAWA, Kyoto (JP)

(73) Assignee: OMRON HEALTHCARE CO., LTD., Muko-shi (JP)

(21) Appl. No.: 19/183,106

(22) Filed: Apr. 18, 2025

Related U.S. Application Data
(63) Continuation of application No. PCT/JP2023/028712, filed on Aug. 7, 2023.

(30) Foreign Application Priority Data
Nov. 11, 2022 (JP) 2022-181105

(51) Int. Cl. A61B 5/021; A61B 5/00; A61B 50/20

(52) U.S. Cl. CPC A61B 5/02141; A61B 5/742; A61B 50/20

(57) ABSTRACT

A sphygmomanometer that can be operated with a mobile terminal includes a main unit casing a cuff for applying a pressure to a measurement region of a subject, and a communication unit that communicates wirelessly with the mobile terminal. The main unit casing includes a cuff housing configured to removably house the cuff, and a mobile terminal holder on which the mobile terminal is placed.

### CROSS REFERENCE TO RELATED APPLICATION

This is a continuation application of International Application No.PCT/JP2023/028712, with an International filing date of Aug. 7, 2023, which claims priority of Japanese Patent Application No. 2022-18110 filed on Nov. 11, 2022, the entire content of which is hereby incorporated by reference.

### BACKGROUND

### Technical Field

The present disclosure relates to a coil component. a sphygmomanometer.

### Background Art

The sphygmomanometer described in CN-B-102058404 includes a main unit having a stand for a mobile terminal. The mobile terminal resting on the stand communicates with the main unit, so that the mobile terminal can be used in operating the main unit and to display a blood pressure measured by the main unit. To the main unit, a cuff for applying pressure to a measurement region of a subject is connected via a tube, and the cuff is left outside even when the cuff is not in use.

### SUMMARY

Blood pressures are generally measured not only once, but repeatedly at a certain frequency. With the sphygmomanometer according to CN-B-102058404, although the mobile terminal can be carried with the main unit, in a manner resting on the stand, but the cuff is not suitable to be carried with the main unit, as the cuff is connected to the main unit via a tube and remains exposed even while not in use. With the sphygmomanometer according to Patent Document 1, the cuff remains exposed during the use, too, and is not attractive in the appearance. Therefore, such a sphygmomanometer is often stored so as not to be visible to anyone, without being left outside in the room, while the sphygmomanometer is not in use. Once the sphygmomanometer is put into a storage, it is cumbersome for the user to make the measurement next time. As described above, despite being enabled to operate with a mobile terminal, the sphygmomanometer according to Patent Document 1 has resulted in a less measurement frequency because the sphygmomanometer is not suitable for carrying and has an unattractive appearance.

An object of the present invention is to contribute to ensuring a measurement frequency through improvements in the portability and the appearance of a sphygmomanometer enabled to operate using a mobile terminal.

One aspect of the present invention provides a sphygmomanometer operable by a mobile terminal, the sphygmomanometer including: a main unit casing; a cuff configured to apply a pressure to a measurement region of a subject; and a communication unit configured to communicate wirelessly with the mobile terminal, in which the main unit casing includes a cuff housing configured to removably house the cuff, and a mobile terminal holder on which the mobile terminal is placed.

Both of the cuff housing and the mobile terminal holder are provided to the main unit casing. In other words, the cuff housing and the mobile terminal holder are provided integrally with the main unit casing. Therefore, by housing the cuff in the cuff housing and placing the mobile terminal on the mobile terminal holder, the main unit casing, the cuff, and the mobile terminal can be put together as one unit and carried at once. Furthermore, because the cuff is housed in the cuff housing without being left exposed while the cuff is not in use, the sphygmomanometer also has excellent appearance. It is therefore not necessary to put the sphygmomanometer in a storage so as not to be exposed to people's eyes, and the sphygmomanometer can be left in the room. As described above, because the sphygmomanometer can be easily carried and has an excellent appearance, in addition to being operable using the mobile terminal, the sphygmomanometer can contribute to ensuring the frequency of measurements.

The mobile terminal holder may include: a support wall surface that supports a lower part of the mobile terminal resting in an inclined orientation; the cuff housing may support an upper part of the mobile terminal resting in an inclined orientation; and a part between the lower part and the upper part of the mobile terminal resting on the mobile terminal holder may be spaced apart from the main unit casing.

The mobile phone resting on the mobile terminal holder in the inclined orientation is supported in such a manner that a part between the upper part and the lower part is spaced apart from the main unit casing. In other words, the mobile phone is leaned against the main unit casing in such a manner that a part between the upper part and the lower part is spaced apart from the main unit casing. Therefore, even if an accessory such as a ring is attached to the mobile terminal, the mobile terminal is allowed to rest stably.

The mobile terminal holder may include a protrusion, and a wall surface of the protrusion on a side facing the cuff housing may serve as a support wall surface.

The mobile terminal holder may include a groove, and a groove wall surface of the groove may serve as the support wall surface, the groove wall surface being a surface on an opposite side of the cuff housing.

A detection element configured to detect that the mobile terminal is placed on the mobile terminal holder may be provided.

The detection element may be an infrared light-emitting and light-receiving element configured to detect that the mobile terminal is placed, on the support wall surface and a part facing the support wall surface of the main unit casing.

The height of the support wall surface may be set to 8 mm or less.

By setting the height of the support wall surface in this manner, the operations to the touch panel, the button, the sensor, and the like on the lower side of the mobile terminal resting on the mobile terminal holder is not obstructed.

The mobile terminal holder may have both ends with open clearances on lateral sides or a front side of the main unit casing.

With this configuration, a foldable mobile terminal or a mobile terminal fitted with a folio cover is allowed to rest on the mobile terminal holder, with the mobile terminal or the folio cover opened.

The mobile terminal holder may be provided on a right side, in a front view of the main unit casing.

By providing the mobile terminal holder on the right side in the front view, the operability of the mobile terminal resting on the mobile terminal holder is improved for a right-handed subject.

The sphygmomanometer may include a first indicator configured to indicate a measurement result, and an operation unit configured to receive an instruction including an instruction related to an operation of the cuff from a subject, and the first indicator and the operation unit may be each provided at a position hidden behind the mobile terminal resting on the mobile terminal holder of the main unit casing.

While the mobile terminal is resting on the mobile terminal holder, the first indicator and the operation unit of the sphygmomanometer are hidden behind the mobile terminal, so that the subject can operate the sphygmomanometer using the mobile terminal without becoming confused.

The cuff housing may have a tubular shape having an opening on a top, and the opening may be openably closed by a lid.

The cuff housing may include a slit for inserting a tube connected to the cuff.

A connector attachment for attaching a connector for connecting the tube to air piping provided to the main unit casing may be provided inside the cuff housing, and a bottom end of the slit may be positioned lower than the connector attachment.

By setting the bottom end of the slit to a position lower than the connector attachment, even if water enters the cuff housing while the lid is open, the entered water is discharged to the outside of the cuff housing through the bottom end of the slit, so that it is possible to prevent the water from getting into the sphygmomanometer through the connector attachment.

The main unit casing may include an electric component housing in which an electric component including a pump for supplying fluid to the cuff is housed, in a part on a side other than a bottom part of the cuff housing.

With this configuration, it is possible to keep the height of the cuff housing low.

The second indicator for displaying an indication for encouraging the user to measure the blood pressure may be provided at a position different from the position of the first indicator.

The sphygmomanometer according to the present invention can be operated with a mobile terminal, is excellent in portability and appearance, and can contribute to ensuring the measurement frequency.

### BRIEF DESCRIPTION OF DRAWINGS

FIG. 1 is a perspective view of a sphygmomanometer according to an embodiment of the present invention (with a lid closed).

FIG. 2 is a perspective view of the sphygmomanometer according to the embodiment of the present invention (with the lid opened).

FIG. 3 is a perspective view of the sphygmomanometer according to the embodiment of the present invention during the use.

FIG. 4 is a plan view of the sphygmomanometer according to the embodiment of the present invention.

FIG. 5 is a front view of the sphygmomanometer according to the embodiment of the present invention.

FIG. 6 is a right side view of the sphygmomanometer according to the embodiment of the present invention.

FIG. 7 is a left side view of the sphygmomanometer according to the embodiment of the present invention.

FIG. 8 is a rear view of the sphygmomanometer according to the embodiment of the present invention.

FIG. 9 is a cross-sectional view taken along line IX-IX in FIG. 5.

FIG. 10 is a block diagram of the sphygmomanometer according to the embodiment of the present invention.

FIG. 11 is a perspective view illustrating mechanisms housed inside a main unit casing.

FIG. 12 is a perspective view of the sphygmomanometer according to the embodiment of the present invention (with a smartphone resting thereon).

FIG. 13 is a perspective view of the sphygmomanometer according to the embodiment of the present invention (with a smartphone fitted with a folio cover resting thereon).

FIG. 14 is a right side view of the sphygmomanometer according to the embodiment of the present invention (with a smartphone resting thereon).

FIG. 15 is a cross-sectional view that is the same as FIG. 9 but with a smartphone resting.

FIG. 16 is a perspective view of a sphygmomanometer according to a first modification of the present invention.

FIG. 17 is a plan view of the sphygmomanometer according to the first modification of the present invention.

FIG. 18 is a front view of the sphygmomanometer according to the first modification of the present invention.

FIG. 19 is a schematic cross-sectional view taken along line XIX-XIX in FIG. 18.

FIG. 20 is a perspective view of a sphygmomanometer according to a second modification of the present invention.

FIG. 21 is a plan view of the sphygmomanometer according to the second modification of the present invention.

FIG. 22 is a front view of the sphygmomanometer according to the second modification of the present invention.

FIG. 23 is a schematic cross-sectional view taken along line XXIII-XXIII in FIG. 22.

### DETAILED DESCRIPTION

An embodiment of the present invention will now be described with reference to the accompanying drawings.

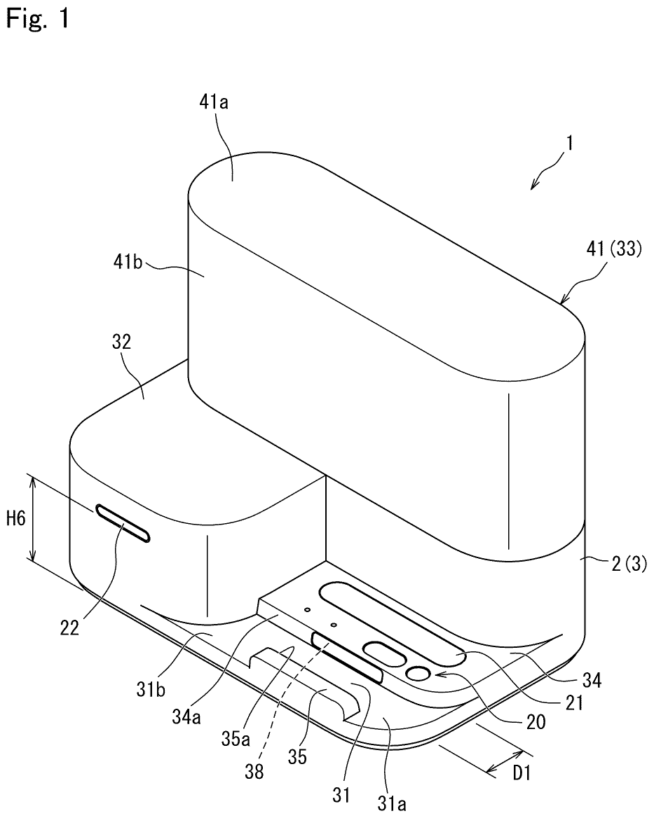

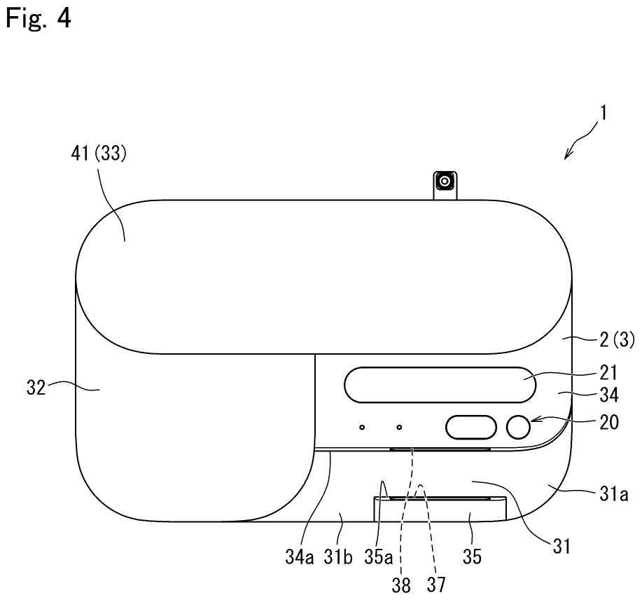

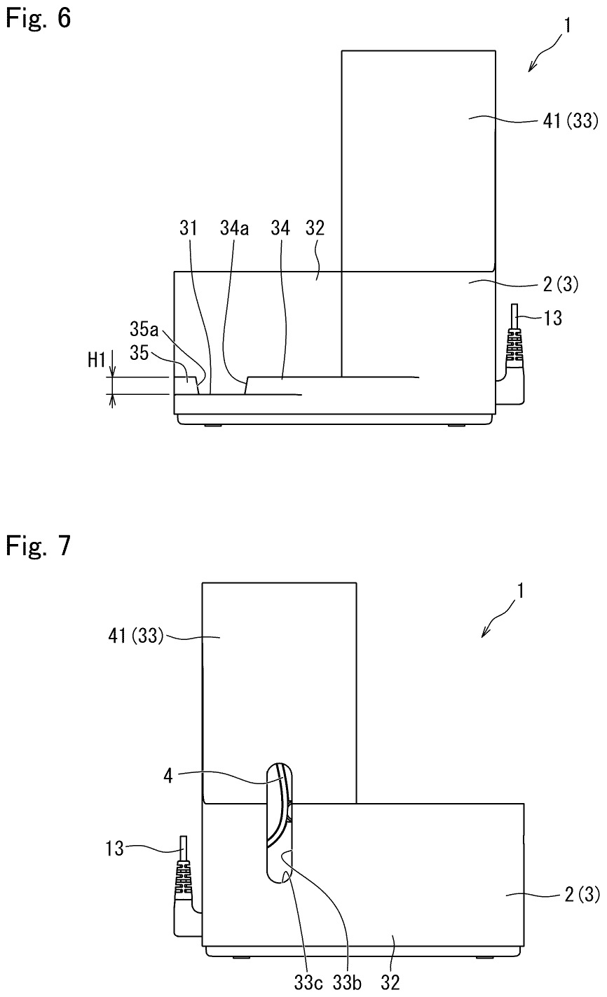

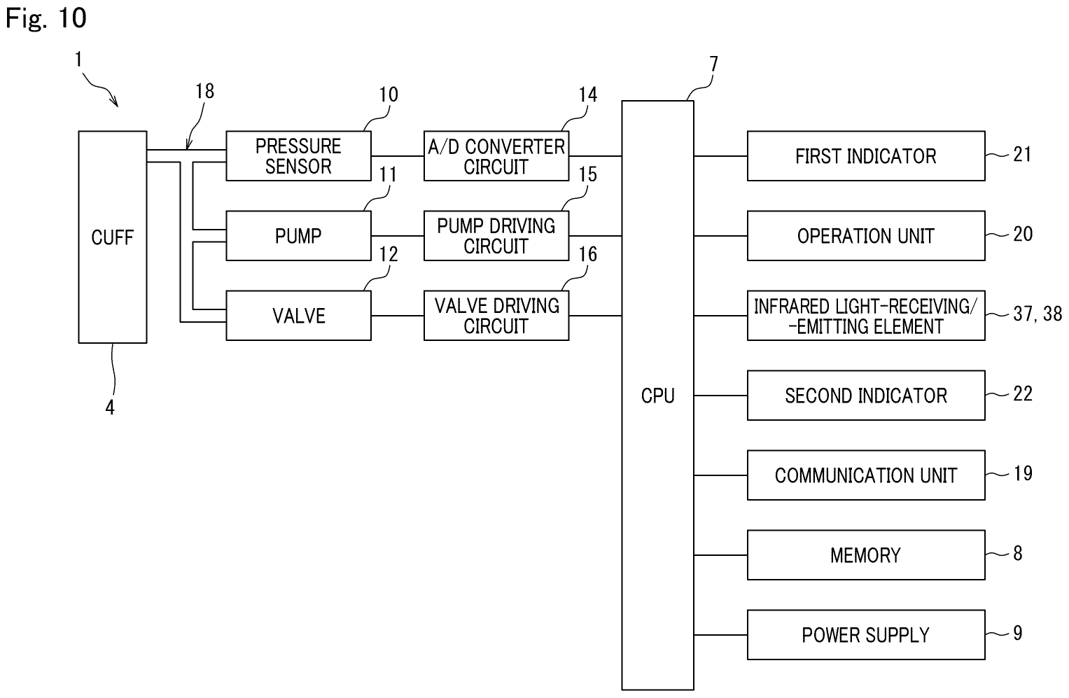

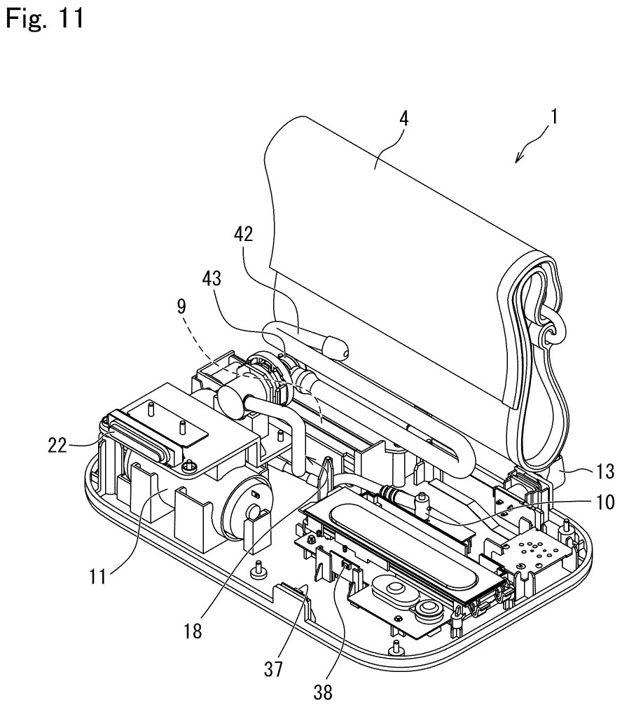

Referring to FIGS. 1 to 9, a sphygmomanometer 1 according to the present embodiment includes a sphygmomanometer main unit 2 and a cuff 4. The sphygmomanometer main unit 2 includes a main unit casing 3 and various electrical components, such as a pump 11, (illustrated in FIGS. 10 and 11) housed inside the main unit casing 3. The cuff 4 has a band-like shape and can be wound in a tubular shape, and is used in a manner wound around an upper arm of a subject. The sphygmomanometer 1 can be operated using a smartphone 6, which is an example of a mobile terminal, by attaching the smartphone 6 to the sphygmomanometer 1, as will be described in detail. Another example of the mobile terminal other than a smartphone is a pad-like mobile information terminal, and any mobile terminal capable of communicating with the sphygmomanometer 1 over the BLE protocol, for example, may be used, without any particular limitation to a specific configuration. As will be described in detail later, the sphygmomanometer 1 can store the cuff 4 inside the cuff housing 33 provided to the main unit casing 3, in a manner allowing the cuff 4 to be taken out.

Also referring to FIGS. 10 and 11, the main unit casing 3 houses a CPU 7, a memory 8, a power supply 9 (secondary battery in the present embodiment), a pressure sensor 10, a pump 11, and a valve 12. In the present embodiment, the power supply 9 is charged by a commercial power supply via a power cable 13. It is however also possible to charge the power supply 9 wirelessly, by disposing the sphygmomanometer 1 in a charging cradle, for example. Housed in the main unit casing 3 are an A/D converter circuit 14 for converting an output of the pressure sensor 10 from an analog signal to a digital signal, a pump driving circuit 15 for driving the pump 11, and a valve driving circuit 16 for driving the valve 12. The pressure sensor 17, the pump 11, and the valve 12 are connected to a bladder (not illustrated) included in the cuff 4 via air piping 18, in a manner allowing a fluid to flow therethrough. A communication unit 19 for communicating wirelessly with the smartphone 6 is also housed inside the main unit casing 3.

The main unit casing 3 is provided with an operation unit 20 for receiving instructions including an instruction related to the operation of the cuff 4 from the subject, a first indicator 21 for displaying information such as measurement results, and a second indicator 22 for making a display for encouraging the user to measure the blood pressure. In the present embodiment, the operation unit 20 includes a plurality of push buttons. The first indicator 21 is provided as a liquid crystal display panel, and the second indicator 22 is provided as an LED lamp.

The CPU 7 controls the operations of the entire sphygmomanometer 1 comprehensively. In particular, the CPU 7 executes a blood pressure measurement by causing the pump driving circuit 15 and the valve driving circuit 16 to operate the pump 11 and the valve 12, respectively, in accordance with an input from the subject, received via the operation unit 20 or the smartphone 6, and a control program stored in the memory 8, so as to control the pressure (cuff pressure) of the air bladder inside the cuff 4 by supplying the air into the bladder in the cuff 4, for example. The cuff pressure is detected by the pressure sensor 17. The CPU 7 also presents the measurement result on the first indicator 21.

Referring to FIGS. 1 to 9, the main unit casing 3 includes a smartphone holder 31 where the smartphone 6 is placed, on the front right half of the main unit casing 3 in the front view, an electric component housing 32 on the front left half of the main unit casing 3 in the front view, and a cuff housing 33 stretching across the entire width of the rear side of the main unit casing 3 in the front view. Between the smartphone holder 31 and the cuff housing 33, a panel unit 34 having the operation unit 20 and the first indicator 21 on the top surface is provided. The cuff housing 33 is connected to the panel unit 34 on the rear side in the front view.

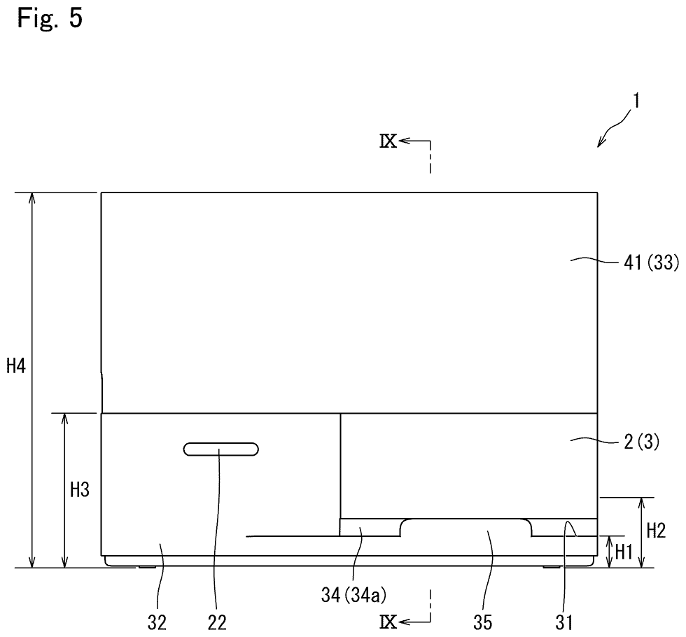

As illustrated in FIG. 5, the height H1 of the smartphone holder 31 is the smallest, and the height H2 of the panel unit 34 is slightly greater than the height H1 of the smartphone holder 31. The height H3 of the electric component housing 32 is significantly higher than the height H1 of the smartphone holder 31, and the height H4 of the cuff housing 33 is significantly higher than the height H3 of the electric component housing 33. The height H4 of the cuff housing 33 corresponds to the height of the entire main unit casing 3.

The electric component housing 32 has a sufficient height H3, occupies about half the width of the main unit casing 3 in a front view, and has a depth exceeding a half of the main unit casing 3. In other words, the electric component housing 32 has a sufficient volume. A large number of electric components including the pump 11, which is the largest electric component, are thus housed inside the electric component housing 32. In this manner, components to be housed in other parts of the main unit casing 3, that is, components to be housed inside the bottom part of the cuff housing 33, the smartphone holder 31, and the panel unit 34 can be minimized. In particular, by minimizing the number of electrical components to be housed in the bottom part of the cuff housing 33, the height H4 of the cuff housing 33, that is, the overall height of the main unit casing 3 can be reduced. Furthermore, the height H1 of the smartphone holder 31 and the height H2 of the panel unit 34 can also be kept small.

The smartphone holder 31 will now be explained.

The smartphone holder 31 has a flat top surface, and includes a clearance 31a that is provided to the right end in a front view and opens to the right end of the main unit casing 3, and a clearance 31b that is provided to the front end in the front view and that opens to the front end of the main unit casing 3.

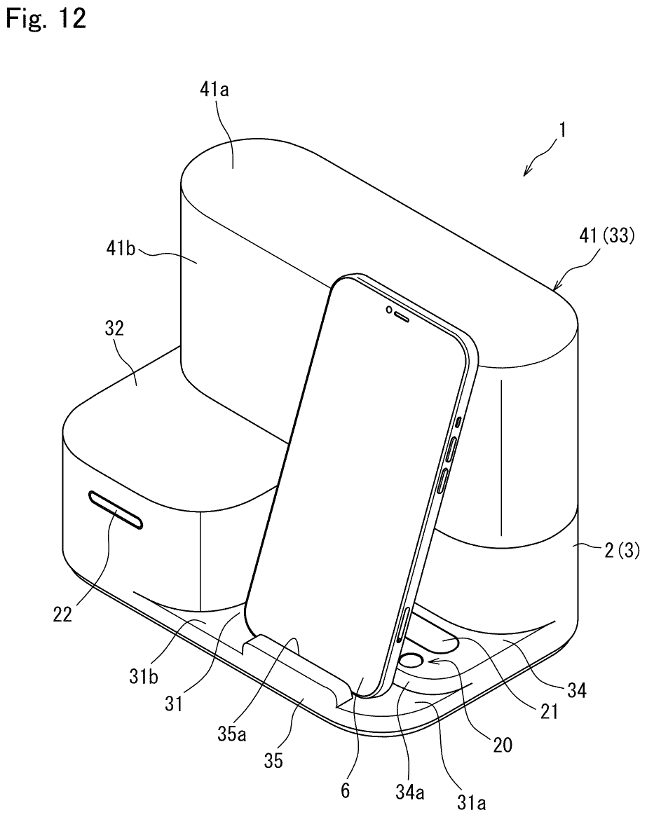

The smartphone holder 31 includes a protrusion 35 having an elongated rectangular parallelepiped shape and extending in the width direction of the main unit casing 3 along the front side in a front view. Referring also to FIGS. 12 to 14, the protrusion 35 supports the lower part of the smartphone 6, and the cuff housing 33 supports the upper part of the smartphone 6 from the rear side. In this manner, the smartphone 6 rests on the smartphone holder 31 in a manner inclined rearwards. More specifically, the lower part of the smartphone 6 that is inclined rearwards is supported by a wall surface of the protrusion 35, the wall surface being on the side facing the cuff housing 33, so that the smartphone 6 does not move frontwards, and the upper part of the smartphone 6 that is inclined rearwards is supported by the cuff housing 33 so that the smartphone 6 does not become inclined further rearwards. In this embodiment, the smartphone 6 is supported by the front upper edge of the cuff housing 33 (more specifically, a lid 41, to be described later) in a plan view. However, when the smartphone 6 is smaller than that used in this embodiment, the front side of the cuff housing 33 (more specifically, the lid 41, to be described later) supports the top end of the smartphone 6. Even when the lid 41 is removed, the upper part of the smartphone 6 remains supported by the cuff housing 33 in the same orientation, as will be described later.

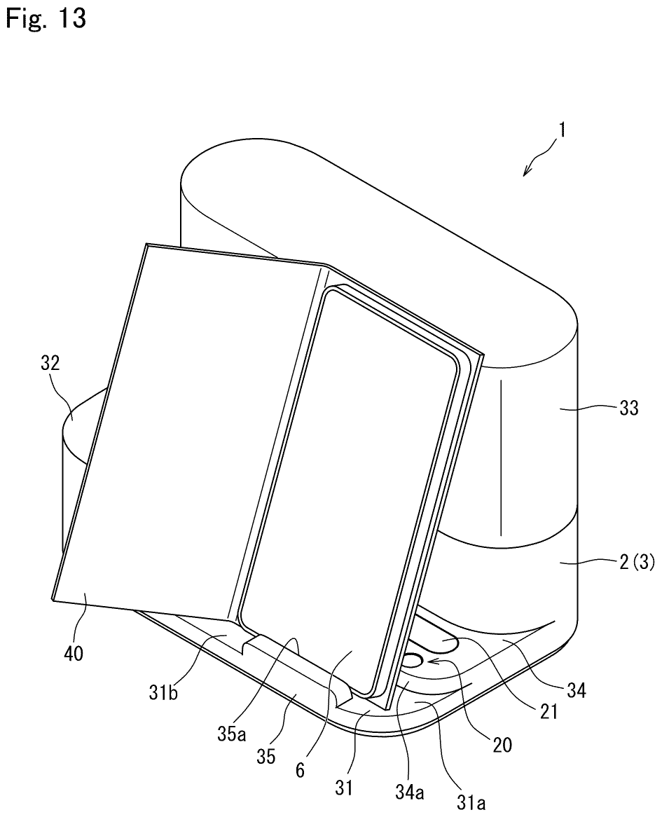

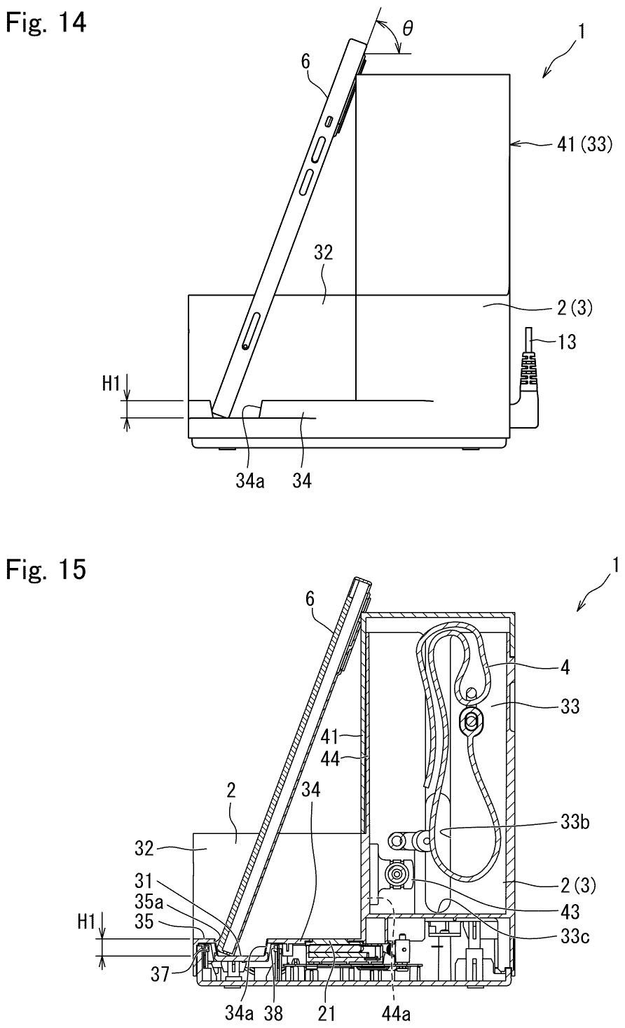

The angle θ (indicated in FIG. 14) by which the smartphone 6 rests on the smartphone holder 31 tilts rearwards is not limited to a particular angle, as long as visibility can be ensured, but may be set to 65 degrees or more and 70 degrees or less, for example.

As most clearly illustrated in FIGS. 12, 14, and 15, on the smartphone 6 resting on the smartphone holder 31 in a manner inclined rearwards, the part between the upper and the lower parts is spaced apart from the main unit casing 3. In other words, the smartphone 6 is leaned against the main unit casing 3, with the part between the upper part and the lower part positioned spaced apart from the main unit casing 3. Therefore, even if an accessory such as a ring is attached to the rear surface of the smartphone 6, the smartphone 6 can rest stably on the smartphone holder 31.

Referring to FIGS. 1, 2, 9 to 11, and 15, an infrared light-emitting element 37 is provided to a part of a support wall surface 35a of the protrusion 35 provided to the smartphone holder 31, and an infrared light-receiving element 38 is provided to a part of the main unit casing 3, the part being a part facing the support wall surface 35a, that is, on a front surface 34a of the panel unit 34. While the sphygmomanometer 1 is receiving power supply, the infrared light-emitting element 37 keeps outputting infrared light toward the infrared light-receiving element 38. When the smartphone 6 is not placed on the smartphone holder 31, as illustrated in FIG. 1, for example, the infrared light-receiving element 38 keeps receiving the infrared light. While the smartphone 6 is resting on the smartphone holder 31, as illustrated in FIG. 12, for example, the smartphone 6 blocks the infrared light from the infrared light-emitting element 37, so that the infrared light-receiving element 38 does not receive the infrared light. When the CPU 7 detects that the infrared light-receiving element 38 stops receiving infrared light, the CPU 7 causes the communication unit 19 to start communicating with the smartphone 6, temporarily stops the functions of the first indicator 21 and the operation unit 20, and switches the functions such as those of receiving the instructions from the subject and displaying the measurement result to the smartphone 6.

The way for detecting whether the smartphone 6 is on the smartphone holder 31 is not limited to the combination of the infrared light-emitting element 37 and the infrared light-receiving element 38, as disclosed in this embodiment. An alternative configuration is, for example, a switch (such as a measurement button on a conventional sphygmomanometer) triggered by becoming physically pushed by the smartphone 6 as the smartphone 6 is disposed on the smartphone holder 31. In such a case, the sphygmomanometer 1 may be configured to measure the blood pressure when the switch is pressed by the smartphone 6. It is also possible to detect that the smartphone 6 has been placed on the smartphone holder 31 using a capacitive proximity sensor, or near field communication (NFC) between the sphygmomanometer 1 and the smartphone 6.

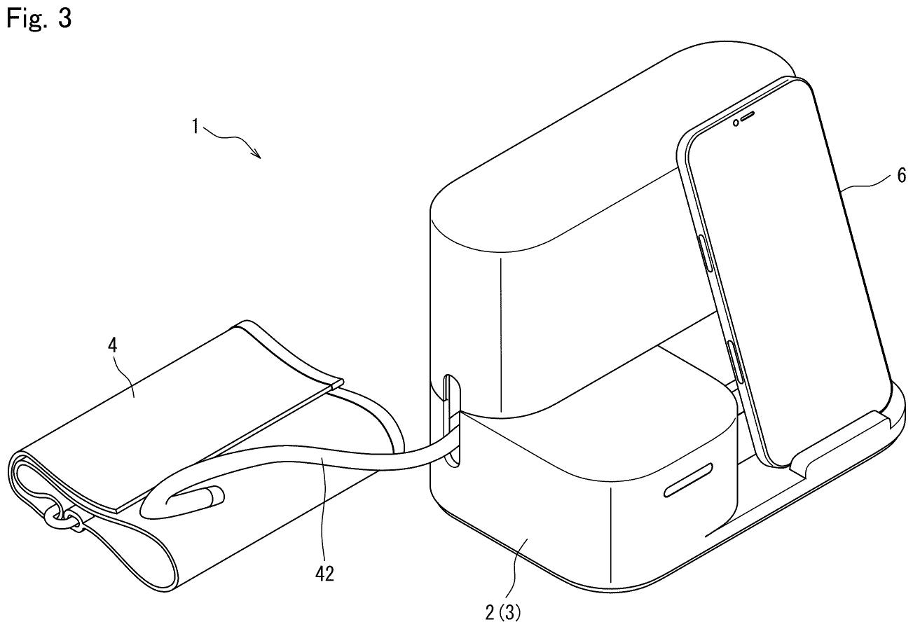

Referring to FIGS. 3 and 12 to 15, the operation unit 20 and the first indicator 21 provided on the panel unit 34 of the main unit casing 3 are at positions to become hidden by the smartphone 6 resting on the smartphone holder 31, when the smartphone 6 is placed. When the smartphone 6 is resting on the smartphone holder 31, the operation unit 20 and the first indicator 21 are hidden thereby, so that the subject can operate the sphygmomanometer 1 using the smartphone 6 without becoming confused.

As described above, open clearances 31a, 31b are provided to both respective ends of the smartphone holder 31. Therefore, for example, a smartphone 6 with a folio cover 40 fitted thereto, as illustrated in FIG. 13, or a foldable smartphone 6 can be placed, in a manner opened, on the smartphone holder 31.

Referring to FIG. 1, although there is no particular limitation to the distance D1 between the support wall surface 35a of the protrusion 35 provided to the smartphone holder 31 and the front surface 34a of the panel unit 34 facing the support wall surface 35a, the distance D1 is preferably set so as to enable even a thick smartphone 6 or the smartphone 6 housed in a case can be placed on the smartphone holder 31.

Referring to FIGS. 14 and 15, the support wall surface 35a and the front surface 34a are inclined to stabilize the lower end of the smartphone 6 while the smartphone is resting on the smartphone holder 31.

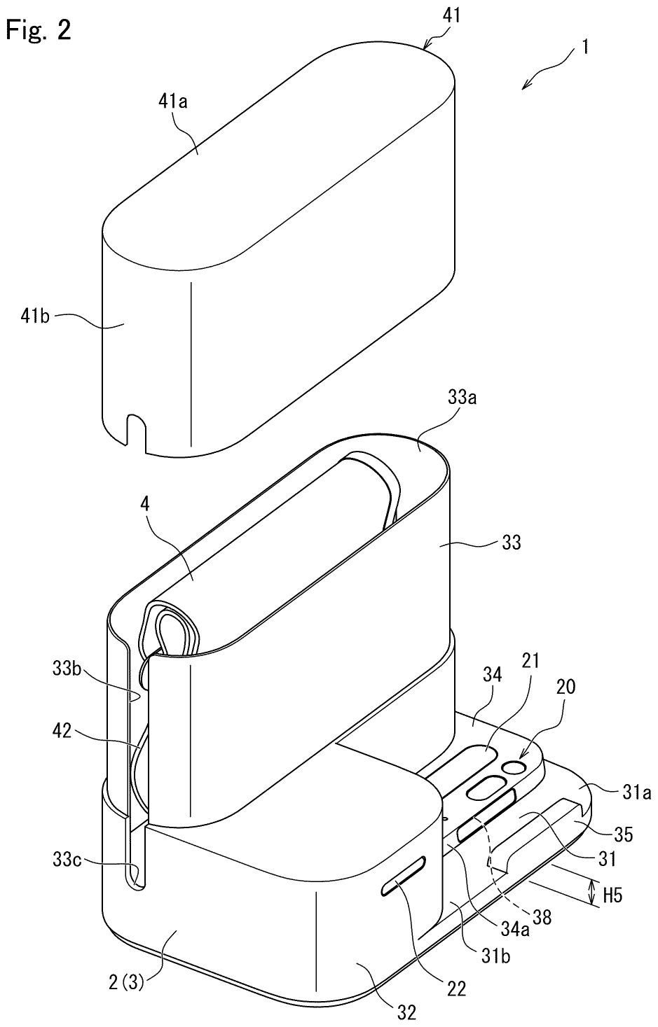

Referring to FIG. 2, the height of the protrusion 35 provided to the smartphone holder 31, that is, the height H5 of the support wall surface 35a is preferably set to 8 mm or less. By setting the height H5 of the support wall surface 35a in the manner described above, the protrusion 35 does not obstruct operations of a touch panel, a button, a sensor, and the like on a lower part of the smartphone 6 resting on the smartphone holder 31.

As mentioned earlier, the smartphone holder 31 is provided on the right side in front view of the main unit casing 3. By providing the smartphone holder 31 on the right side, the operability of the smartphone 6 held on the smartphone holder 31 is improved for a right-handed subject.

The cuff housing 33 will now be explained.

Referring to FIGS. 1 to 9, the cuff housing 33 according to this embodiment has a tubular shape having a track-shaped opening 33a on the top in a plan view, and the opening 33a is closed by a lid 41 that is removable from the cuff housing 33. The lid 41 includes a flat top wall 41a and a peripheral wall 41b extending from the periphery of the top wall 41a. The lid 41 is not limited to a removable lid, as long as the opening 33a can be closed in an openable manner, and may be a sliding lid, a hinged lid, or a lid connected to the cuff housing 33 via a thin and flexible wall, for example.

Referring to FIGS. 2, 3, 7, and 9, a slit 33b extending in the vertical direction is provided on a right side of the cuff housing 33 in a front view. The slit 33b is for passing the tube 42 having one end connected to the cuff 4, when the cuff 4 is taken out from the cuff housing 33. The other end of the tube 42 is connected to a connector 43. The tube 42 is connected to the air piping 18 via the connector 43. As illustrated in FIG. 9, the connector 43 is attached to a connector attachment 44a that is a through hole provided to a partitioning wall 44 between the cuff housing 33 and the electric component housing 32. The bottom end 33c of the slit 33b is positioned lower than the connector attachment 44a. Therefore, even if water enters the cuff housing 33 while the lid 41 is open, the entered water is discharged to the outside of the cuff housing 33 through the bottom end 33c of the slit 33b, so that it is possible to prevent the water from getting into the sphygmomanometer 1, more specifically, into the electric component housing 32, through the connector attachment 44a.

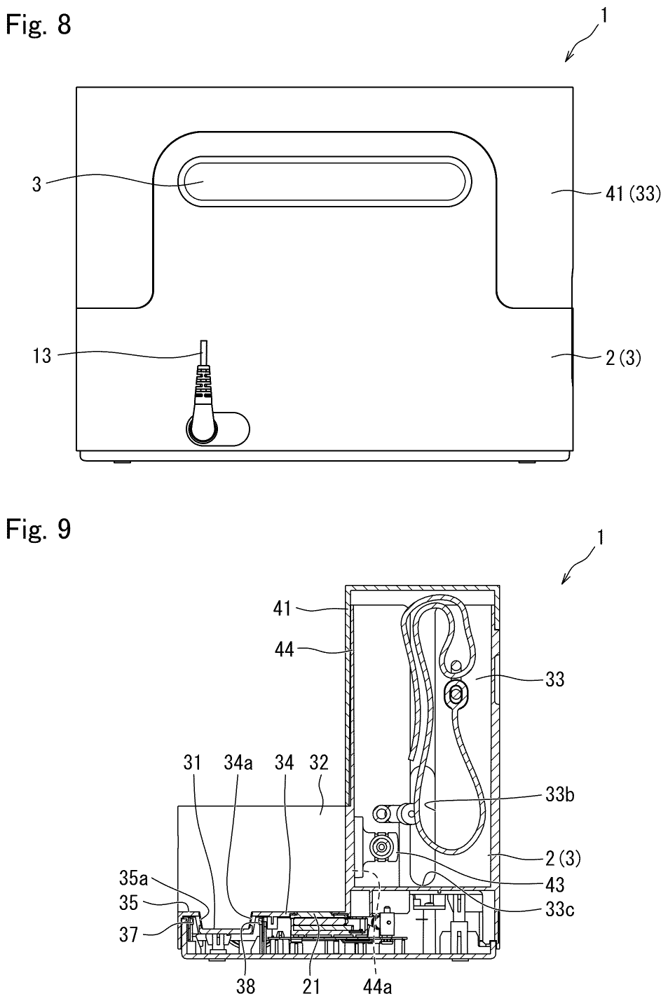

Both of the cuff housing 33 and the smartphone holder 31 are provided to the main unit casing 3. In other words, the cuff housing 33 and the smartphone holder 31 are provided integrally to the main unit casing 3. Therefore, it is possible to carry the main unit casing 3, the cuff 4, and the smartphone 6 easily, with the cuff 4 housed inside the cuff housing 33 and the smartphone 6 resting on the smartphone holder 31, that is, with all of the main unit casing 3, the cuff 4, and the smartphone 6 put together as one unit. In particular, as illustrated in FIG. 8, a recess 3a for hooking fingers is provided on the rear surface of the main unit casing 3.

In addition, because the cuff 4 and the tube 42 are housed inside the cuff housing 33 and do not remain exposed outside while not in use, the appearance is excellent. It is therefore not necessary to put the sphygmomanometer in a storage so as not to be exposed to people's eyes, and the sphygmomanometer can be left in the room. As described above, the sphygmomanometer 1 according to the present embodiment can be operated with the smartphone 6, can be carried easily, has an excellent appearance, and can contribute to ensuring the measurement frequency.

Referring to FIG. 1, in the present embodiment, the second indicator 22 for making a display for encouraging the user to measure the blood pressure is provided at a position different from the first indicator 21 provided on the panel unit 34, more specifically, provided to the front surface of the electric component housing 32. In the present embodiment, the second indicator 22 is provided to the upper part of the front surface of the electric component housing 32, more specifically, at a height H6 of ½ the distance between the lowermost end to the uppermost end of the main unit casing 3 or more. This is because the visibility of the second indicator 22 is improved, and the subject is allowed to notice the display for encouraging the blood pressure measurement more easily. The position and height at which the second indicator 22 is provided are not limited to a particular position and height, as long as the visibility is ensured.

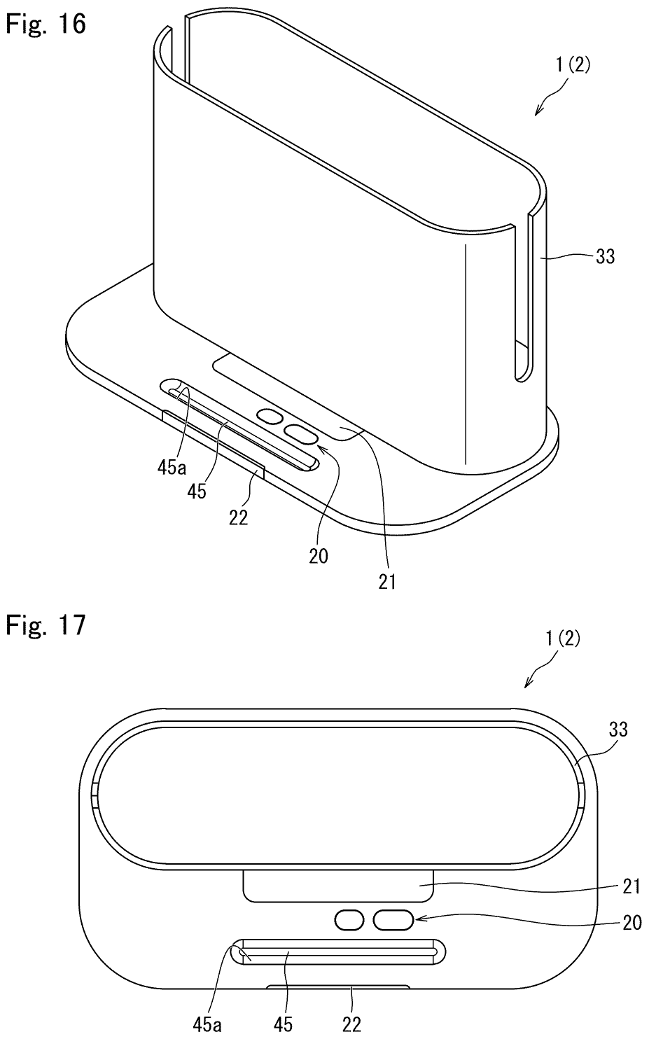

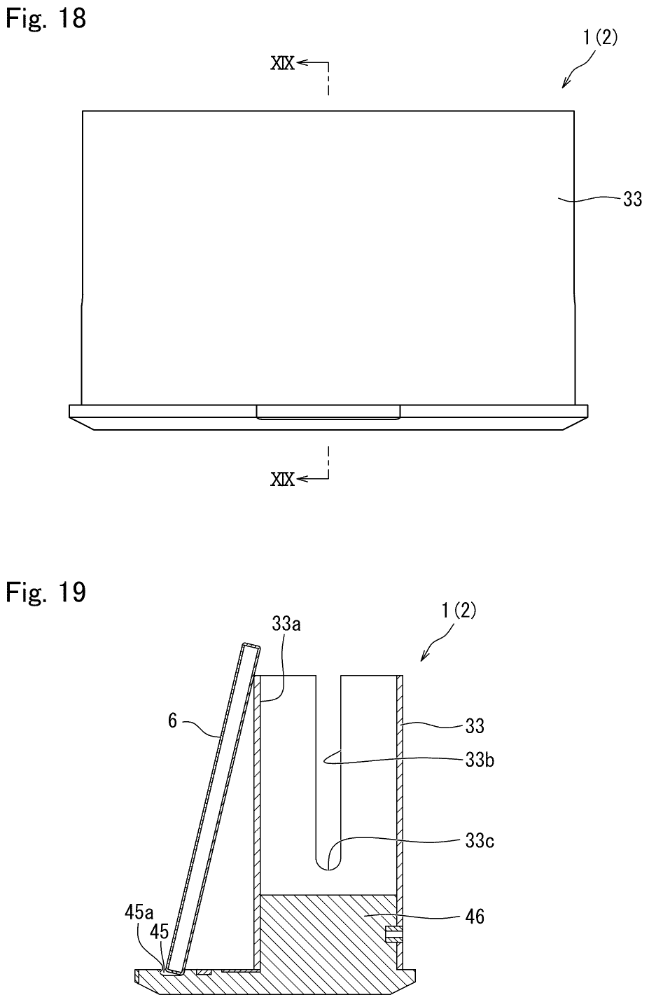

FIGS. 16 to 19 illustrate a first modification of the present embodiment. In this modification, the smartphone holder 31 includes, instead of the protrusion 35 (see, for example, FIG. 1), a groove 45 extending in the width direction of the main unit casing 3, and a groove wall of the groove 45 on the side opposite to the cuff housing 33 serves as a support wall surface 45a. In the first modification, electric components such as a pump are housed under the cuff housing 33 (indicated by the reference numeral 46 in FIG. 19), and the electric component housing 32 (see, for example, FIG. 1) is omitted. By housing the electrical components under the cuff housing 33, it is possible to reduce the size of the main unit casing 3 in the depth direction.

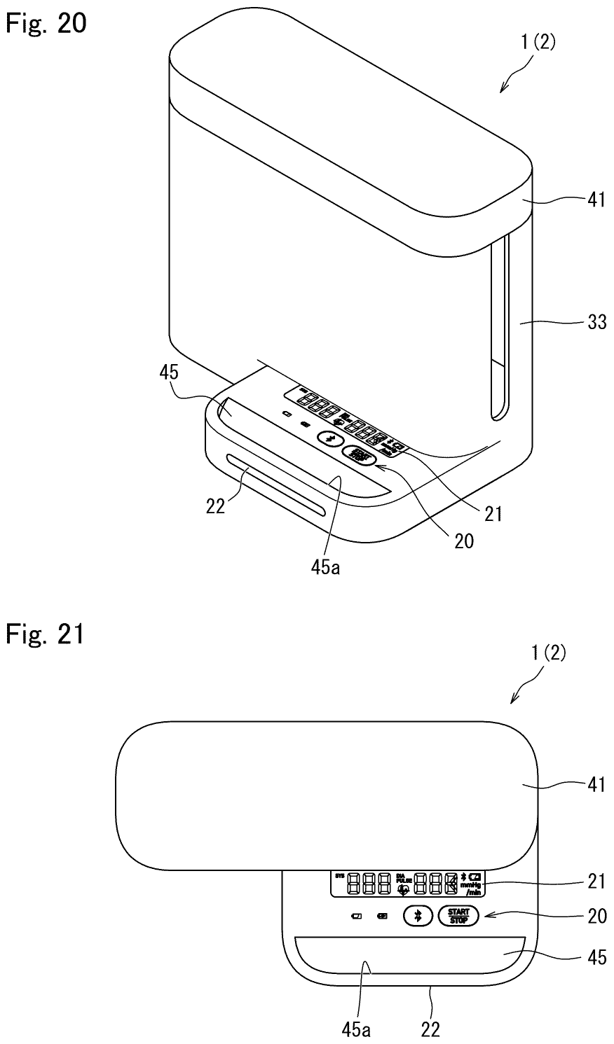

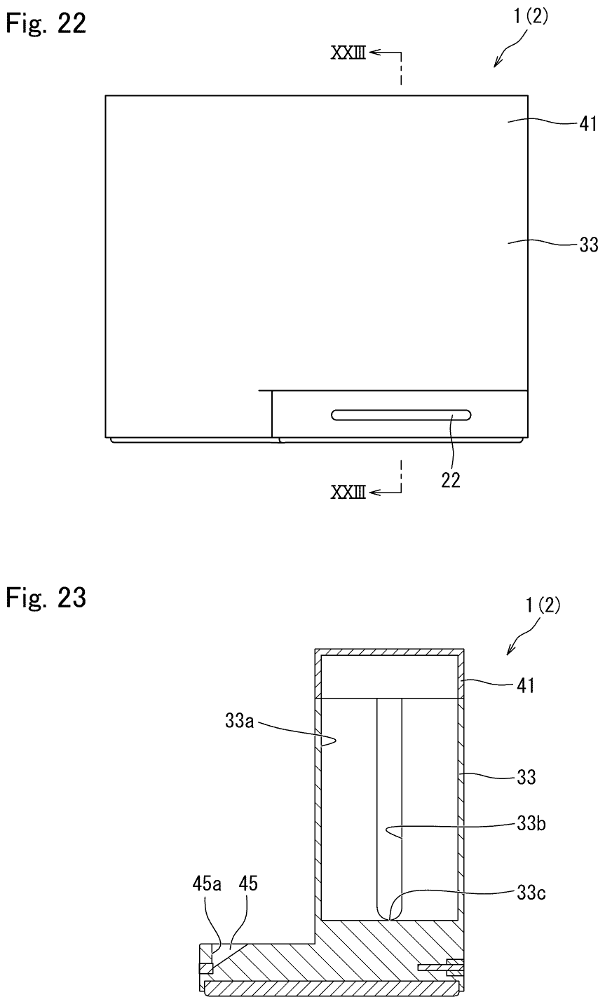

FIGS. 20 and 21 illustrate a second modification of the present embodiment. In this modification, the smartphone holder 31 includes, instead of the protrusion 35 (for example, see FIG. 1), a groove 45 provided on the front side of the panel unit 34 and extending in the width direction of the main unit casing 3, and a groove wall of the groove 45 on the side opposite to the cuff housing 33 serves as the support wall surface 45a. In the second modification, electric components such as the pump are housed inside the panel unit 34, and the electric component housing 32 (for example, see FIG. 1) is omitted. In this manner, it is possible to reduce the height of the main unit casing 3 also by housing the electric components inside the panel unit 34.

The embodiments described above are examples, and various modifications can be made within the scope not departing from the scope of the present invention. Each of the plurality of embodiments described above can be implemented independently, but the embodiments may also be combined. In addition, various features in different embodiments may be implemented independently, but features in different embodiments may also be combined.

What is claimed is:

1. A sphygmomanometer operable by a mobile terminal, the sphygmomanometer comprising:
a main unit casing;
a cuff configured to apply a pressure to a measurement region of a subject; and
a communication unit configured to communicate wirelessly with the mobile terminal, wherein
the main unit casing includes
a cuff housing configured to removably house the cuff, and
a mobile terminal holder on which the mobile terminal is placed
the mobile terminal holder comprises support wall surface that supports a lower part of the mobile terminal resting in an inclined orientation,
the cuff housing supports an upper part of the mobile terminal resting in an inclined orientation, and
a part between the lower part and the upper part of the mobile terminal resting on the mobile terminal holder is spaced apart from the main unit casing.

2. The sphygmomanometer according to claim 1, wherein
the mobile terminal holder includes a protrusion, and
a wall surface of the protrusion on a side facing the cuff housing serves as the support wall surface.

3. The sphygmomanometer according to claim 1, wherein the mobile terminal holder includes a groove, and a groove wall surface of the groove serves as the support wall surface, the groove wall surface being a surface on an opposite side of the cuff housing.

4. The sphygmomanometer according to claim 1, further comprising a detection element configured to detect that the mobile terminal is placed on the mobile terminal holder.

5. The sphygmomanometer according to claim 4, wherein the detection element is an infrared light-emitting and light-receiving element configured to detect that the mobile terminal is placed, on the support wall surface and a part facing the support wall surface of the main unit casing.

6. The sphygmomanometer according to claim 1, wherein a height of the support wall surface is set to 8 mm or less.

7. A sphygmomanometer operable by a mobile terminal, the sphygmomanometer comprising:
a main unit casing;
a cuff configured to apply a pressure to a measurement region of a subject; and
a communication unit configured to communicate wirelessly with the mobile terminal, wherein
the main unit casing includes
a cuff housing configured to removably house the cuff, and
a mobile terminal holder on which the mobile terminal is placed
a first indicator configured to indicate a measurement result; and
an operation unit configured to receive an instruction, including an instruction related to an operation of the cuff, from a subject,
wherein the first indicator and the operation unit are each provided at a position hidden behind the mobile terminal resting on the mobile terminal holder of the main unit casing.

8. The sphygmomanometer according to claim 7, comprising a second indicator configured to make a display for encouraging a user to measure a blood pressure, at a position different from the position of the first indicator.
